(19) United States
(12) Patent Application Publication
(10) Pub. No.: US 2004/0087024 A1
(43) Pub. Date: May 6, 2004

(54) Carbohydrate-modified polymers, compositions and uses related thereto

(75) Inventors: Nathalie C. Bellocq, Altadena, CA (US); Jianjun Cheng, Arcadia, CA (US); Mark E. Davis, Pasadena, CA (US); Suzie Hwang Pun, Torrance, CA (US)

Correspondence Address: ROPES & GRAY LLP, ONE INTERNATIONAL PLACE, BOSTON, MA, 02110-2624, US

(73) Assignee: Insert Therapeutics, Inc., Pasadena, CA

(21) Appl. No.: 10/372,723

(22) Filed: Feb. 24, 2003

Related U.S. Application Data
(60) Provisional application No. 60/358,830, filed on Feb. 22, 2002.
(60) Provisional application No. 60/417,747, filed on Oct. 10, 2002.

(51) Int. Cl. A61K 48/00; C08G 63/48; C08G 63/91; C12N 15/85; A61K 31/795

(52) U.S. Cl. 435/455; 525/54.2; 514/44; 424/78.3

(57) ABSTRACT

This application discloses compositions of carbohydrate-modified polymers, such as polyethylenimine modified with cyclodextrin moieties, for carrying drugs and other active agents, such as nucleic acids. Compositions are also disclosed of carbohydrate-modified polymer carriers that release such agents under controlled conditions. The invention also discloses compositions of carbohydrate-modified polymer carriers that are coupled to biorecognition molecules for targeting the delivery of drugs to their site of action.

### RELATED APPLICATION

This application is based on U.S. Provisional Applications Nos. 60/358,830, filed Feb. 22, 2002, and 60/417,747, filed Oct. 10, 2002, the specifications of which are hereby incorporated by reference in their entireties herein.

### BACKGROUND OF THE INVENTION

The transfer of nucleic acids into a given cell is at the root of gene therapy. However, one of the problems is to succeed in causing a sufficient quantity of nucleic acid to penetrate into cells of the host to be treated. One of the approaches selected in this regard has been the integration of the nucleic acid into viral vectors, in particular into retroviruses, adenoviruses or adeno-associated viruses. These systems take advantage of the cell penetration mechanisms developed by viruses, as well as their protection against degradation. However, this approach has disadvantages, and in particular a risk of production of infectious viral particles capable of dissemination in the host organism, and, in the case of retroviral vectors, a risk of insertional mutagenesis. Furthermore, the capacity for insertion of a therapeutic or vaccinal gene into a viral genome remains limited.

In any case, the development of viral vectors capable of being used in gene therapy requires the use of complex techniques for defective viruses and for complementation cell lines.

Another approach (Wolf et al. Science 247, 1465-68, 1990; Davis et al. Proc. Natl. Acad. Sci. USA 93, 7213-18, 1996) has therefore consisted in administering into the muscle or into the blood stream a nucleic acid of a plasmid nature, combined or otherwise with compounds intended to promote its transfection, such as proteins, liposomes, charged lipids or cationic polymers such as polyethylenimine, which are good transfection agents in vitro (Behr et al. Proc. Natl. Acad. Sci. USA 86, 6982-6, 1989; Felgner et al. Proc. Natl. Acad. Sci. USA 84, 7413-7, 1987; Boussif et al. Proc. Natl. Acad. Sci. USA 92, 7297-301, 1995).

As regards the muscle, since the initial publication by J. A. Wolff et al. showing the capacity of muscle tissue to incorporate DNA injected in free plasmid form (Wolff et al. Science 247, 1465-1468, 1990), numerous authors have tried to improve this procedure (Manthorpe et al., 1993, Human Gene Ther. 4,419-431; Wolff et al., 1991, BioTechniques 11, 474-485). A few trends emerge from these tests, such as in particular:

the use of mechanical solutions to force the entry of DNA into cells by adsorbing the DNA onto beads which are then propelled onto the tissues (“gene gun”) (Sanders Williams et al., 1991, Proc. Natl. Acad. Sci. USA 88, 2726-2730; Fynan et al., 1993, BioTechniques 11, 474-485). These methods have proved effective in vaccination strategies but they affect only the top layers of the tissues. In the case of the muscle, their use would require a surgical approach in order to allow access to the muscle because the particles do not cross the skin tissues;

the injection of DNA, no longer in free plasmid form but combined with molecules capable of serving as vehicle facilitating the entry of the complexes into cells. Cationic lipids, which are used in numerous other transfection methods, have proved up until now disappointing, because those which have been tested have been found to inhibit transfection (Schwartz et al., 1996, Gene Ther. 3, 405-411). The same applies to cationic peptides and polymers (Manthorpe et al., 1993, Human Gene Ther. 4, 419-431). The only case of a favourable combination appears to be the mixing of poly(vinyl alcohol) or polyvinylpyrrolidone with DNA. The increase resulting from these combinations only represents a factor of less than 10 compared with DNA injected in naked form (Mumper et al., 1996, Pharmaceutical Research 13, 701-709); and

the pretreatment of the tissue to be injected with solutions intended to improve the diffusion and/or the stability of DNA (Davis et al., 1993, Hum. Gene Ther. 4, 151-159), or to promote the entry of nucleic acids, for example the induction of cell multiplication or regeneration phenomena. The treatments have involved in particular the use of local anaesthetics or of cardiotoxin, of vasoconstrictors, of endotoxin or of other molecules (Manthorpe et al., 1993, Human Gene Ther. 4, 419-431; Danko et al., 1994, Gene Ther. 1, 114-121; Vitadello et al., 1994, Hum. Gene Ther. 5, 11-18). These pretreatment protocols are difficult to manage, bupivacaine in particular requiring, in order to be effective, being injected at doses very close to lethal doses. The preinjection of hyperosmotic sucrose, intended to improve diffusion, does not increase the transfection level in the muscle (Davis et al., 1993).

Other tissues have been transfected in vivo either using plasmid DNA alone or in combination with synthetic vectors (reviews by Cotten and Wagner (1994), Current Opinion in Biotechnology 4, 705; Gao and Huang (1995), Gene Therapy, 2, 710; Ledley (1995), Human Gene Therapy 6, 1129). The principal tissues studied were the liver, the respiratory epithelium, the wall of the vessels, the central nervous system and tumours. In all these tissues, the levels of expression of the transgenes have proved to be too low to envisage a therapeutic application (for example in the liver, Chao et al. (1996) Human Gene Therapy 7, 901), although some encouraging results have recently been obtained for the transfer of plasmid DNA into the vascular wall (Iires et al. (1996) Human Gene Therapy 7,959 and 989). In the brain, the transfer efficiency is very low, likewise in tumours (Schwartz et al. 1996, Gene Therapy 3, 405; Lu et al. 1994, Cancer Gene Therapy 1, 245; Son et al. Proc. Natl. Acad. Sci. USA 91, 12669).

### SUMMARY OF THE INVENTION

In certain embodiments, this invention answers the need for improved transfection methods by providing carbohydrate-modified polycationic polymers, such as carbohydrate-modified poly(ethylenimine) (PEI). In certain embodiments, the invention relates to the novel observation that higher levels of carbohydrate modification (i.e., higher average number of carbohydrate moieties per polymer subunit) reduce the toxicity of polycationic polymers such as poly(ethylenimine), while lower levels of carbohydrate modification are generally more compatible with efficient transfection rates. Accordingly, certain embodiments of the invention provide carbohydrate-modified poly(ethylenimine) wherein the degree of carbohydrate modification is selected so as to provide efficient transfection and reduced toxicity to target cells. In further embodiments, the carbohydrate-modified poly(ethylenimine) polymers of the invention have a linear (unbranched) poly(ethylenimine) backbone. In certain preferred embodiments, the invention provides cyclodextrin-modified polycationic polymers, such as cyclodextrin-modified poly(ethylenimine). In certain embodiments, the invention also provides methods of preparing such polymers. In yet additional embodiments, the invention also provides therapeutic compositions containing a therapeutic agent, such as a nucleic acid (e.g., a plasmid or other vector), and a carbohydrate-modified polymer of the invention. Methods of treatment by administering a therapeutically effective amount of a therapeutic composition of the invention are also described.

Carbohydrates that can be used to modify polymers to improve their toxicity profiles include cyclodextrin (CD), allose, altrose, glucose, dextrose, mannose, glycerose, gulose, idose, galactose, talose, fructose, psicose, sorbose, rhamnose, tagatose, ribose, arabinose, xylose, lyxose, ribulose, xylulose, erythrose, threose, erythrulose, fucose, sucrose, lactose, maltose, isomaltose, trehalose, cellobiose and the like. In certain embodiments, the polymer is modified with cyclodextrin moieties and/or galactose moieties.

In one aspect, the invention relates to a kit comprising a carbohydrate polymer, such as a cyclodextrin-modified polyethylenimine (CD-PEI), as described below, optionally in conjunction with a pharmaceutically acceptable excipient, and instructions for combining the polymer with a nucleic acid for use as a transfection system. The instructions may further include instructions for administering the combination to a patient.

In yet another aspect, the invention relates to a method for conducting a pharmaceutical business by manufacturing a polymer or kit as described herein, and marketing to healthcare providers the benefits of using the polymer or kit in the treatment of a medical condition, e.g., for transfecting a patient with a nucleic acid.

In still a further aspect, the invention provides a method for conducting a pharmaceutical business by providing a distribution network for selling a polymer or kit as described herein, and providing instruction material to patients or physicians for using the polymer or kit to treat a medical condition, e.g., for transfecting a patient with a nucleic acid.

Thus, in one aspect, the invention relates to a polymer comprising poly(ethylenimine) (e.g., a polymer comprising at least about 10 or more contiguous ethylenimine monomers, preferably at least 50 or more such monomers) coupled to carbohydrate moieties, such as cyclodextrin moieties. The poly(ethylenimine) may be a branched or a linear polymer. The cyclodextrin moieties may be covalently coupled to the poly(ethylenimine), or may be linked to the poly(ethylenimine) via inclusion complexes (e.g., the polymer is covalently modified with guest moieties, and the cyclodextrin moieties are coupled through formation of inclusion complexes with these moieties). In certain embodiments, at least a portion of the carbohydrate moieties are coupled to the polymer at internal nitrogens (i.e., nitrogen atoms in the backbone of the polymer, as opposed to primary amino groups at termini of the polymer chain). The polymer may have a structure of the formula:

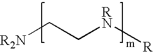

wherein R represents, independently for each occurrence, H, lower alkyl, a moiety including a cyclodextrin moiety, or

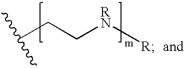

m, independently for each occurrence, represents an integer greater than 10.

The ratio of ethylenimine units to cyclodextrin moieties in the polymer may be between about 4:1 and 20:1, or even between about 9:1 and 20:1.

In another aspect, the invention relates to a polymer comprising a structure of the formula:

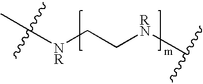

wherein R represents, independently for each occurrence, H, lower alkyl, a moiety including a carbohydrate moiety, or

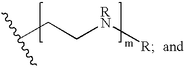

m, independently for each occurrence, represents an integer greater than 10.

In certain embodiments, the polymer is a linear polymer (e.g., R represents H, lower alkyl, or a moiety including a carbohydrate moiety). In certain embodiments, about 3-15% of the occurrences of R represent a moiety including a carbohydrate moiety, preferably other than a galactose or mannose moiety. In certain embodiments, the carbohydrate moieties include cyclodextrin moieties, and may even consist essentially of cyclodextrin moieties. In certain embodiments, about 3-25% of the occurrences of R represent a moiety including a cyclodextrin moiety.

In another aspect, the invention relates to a composition comprising a polymer as described above admixed and/or complexed with a nucleic acid. In yet another aspect, the invention relates to a method for transfecting a cell with a nucleic acid, comprising contacting the cell with such a composition.

In still another embodiment, the invention relates to a kit comprising a polymer as set forth above with instructions for combining the polymer with a nucleic acid for transfecting cells with the nucleic acid.

In a further embodiment, the invention relates to a method of conducting a pharmaceutical business, comprising providing a distribution network for selling a kit or polymer as described above, and providing instruction material to patients or physicians for using the polymer to treat a medical condition.

In still another embodiment, the invention relates to a particles comprising a polymer as described above and having a diameter between 50 and 1000 nm. Such particles may further comprise a nucleic acid, and/or may further comprise polyethylene glycol chains coupled to the polymer through inclusion complexes with cyclodextrin moieties coupled to the polymer.

### BRIEF DESCRIPTION OF THE DRAWINGS

FIG. 1 demonstrates that AD-PEG (an adamantane-polyethylene glycol conjugate) is able to stabilize the CD-PEI polyplexes against salt-induced aggregation when mixed with the polyplexes at a 3:1 ratio (by weight) to the CD-PEI. Addition of PEG even up to 10:1 ratio (by weight) to CD-PEI does not affect the salt stability of the polyplexes.

FIG. 2 shows that AD-PEG is able to stabilize the CD-PEI polyplexes against salt-induced aggregation when mixed with the polyplexes at a 20:1 ratio (by weight) to the CD-PEI. Addition of PEG at 20:1 ratio (by weight) to CD-PEI does not affect the salt stability of the polyplexes.

FIG. 3 compares transfection efficiency of oligonucleotide delivery to cultured cell cells using polymeric delivery vehicles.

FIG. 4 shows in vitro transfection levels using different CD-PEI carriers.

FIG. 5 illustrates how the IC₅₀ of nucleic acids transfected with PEI is increased by over 2 orders of magnitude by heavy grafting of β-cyclodextrin.

FIG. 6 depicts expression of transfected nucleic acid in mouse liver.

FIG. 7 presents results of experiments transfecting hepatoma cells with galactose targeted CD-PEI polymer-based particles containing the luciferase gene.

FIG. 8 shows the correlation between CD-loading and transfection efficiency for CD-bPEI.

FIG. 9 shows the correlation between CD-loading and toxicity for CD-bPEI.

FIG. 10 compares the transfection efficiencies of CD-bPEI and CD-1PEI, and the effect chloroquine has on transfection with these polymers.

FIG. 11 is a photoelectron micrograph of CD-PEI particles.

FIG. 12 demonstrates stabilization of CD-PEI particles against salt-induced aggregation by particle modification with AD-PEG.

FIG. 13 demonstrates the effectiveness of transfections using CD-PEI particles.

### DETAILED DESCRIPTION OF THE INVENTION

I. Overview

Linear and branched poly(ethylenimine)(PEI) are some of the most efficient cationic polymers currently used for in vitro transfections. However, the use of PEI for in vivo applications has been limited due to difficulties in formulation (aggregation in salt) and toxicity of the polymer (Chollet et al. 2001 J of Gene Med). Approaches for improving the formulation conditions of PEI include grafting of the polymer with poly(ethylene glycol) (PEG) and grafting of polyplexes with PEG (Ogris et al. 1999 Gene Ther 6:595-605; and Erbacher et al. 1999 J Gene Med 1:210-222). However, PEI-PEG does not condense DNA into small, spherical particles, and grafting of polyplexes with PEG is difficult to control and to scale-up. Therefore, current PEI systems for in vivo, systemic delivery have not been promising.

Linear cyclodextrin-based polymers (CDPs) have previously been shown to have low toxicity both in vitro (in many different cell lines) and in vivo (Gonzalez et al. 1999 Bioconjugate Chem 10:1068-1074; and Hwang et al. 2001 Bioconjugate Chem 12(2):280-290). We observed that removal of the cyclodextrins from the polymer backbone results in high toxicity of the cationic polymer. This observation led us to conclude that cyclodextrin is able to reduce the toxicity of cationic polymers. In certain embodiments, the present invention is directed to the development of a new method of using cyclodextrins in cationic, cyclodextrin-based polymers to impart stability and targeting ability to polyplexes formed from these polymers.

Since the current linear CDPs transfect poorly into mammalian cell lines (<2% transfection), cyclodextrin-modified polymers of the invention combine the good qualities of the PEI (efficient chloroquine-independent transfection) with the good qualities of the cyclodextrin-based polymers (low toxicity and ability to modify and stabilize the polyplexes). Therefore, as described below, cyclodextrin-grafted polyethylenimine polymers were synthesized and tested. Accordingly, in certain embodiments, preferred carbohydrate-modified polymers of the invention are cyclodextrin-modified polymers, such as cyclodextrin-modified poly(ethylenimines).

The present invention is generally related to a composition comprising carbohydrate-modified polycationic polymers and nucleic acid. In various embodiments, the nucleic acid may be an expression construct, e.g., including a coding sequence for a protein or antisense, an antisense sequence, an RNAi construct, an siRNA construct, an oligonucleotide, or a decoy, such as for a DNA-binding protein.

In certain embodiments, the present compositions have several advantages over other technologies. Most technologies either have high transfection and high toxicity (PEI, Lipofectamine) or low transfection and low toxicity (linear CDPs, other cationic degradable polymers). However, the polymers disclosed herein, such as CD-PEI, have high transfection and low toxicity in vivo. Galactosylated and mannosylated PEI have also been demonstrated to have high transfection with lower toxicity than unmodified PEI, but these polymers do not have any stabilization ability and is likely to aggregate in vivo. The carbohydrate-modified polymers disclosed herein are readily adaptable for in vivo applications via the inclusion-complex modification technology. This would allow for stabilization and targeting of these polyplexes. In addition, the method of carbohydrate modification described herein can increase the IC₅₀ by ˜100-fold, whereas the galactose- and mannose-modified PEI's increase IC₅₀'s only around 10-20 fold.

II. Definitions

For convenience, certain terms employed in the specification, examples, and appended claims are collected here.

The term “ED₅₀” means the dose of a drug that produces 50% of its maximum response or effect.

An “effective amount” of a subject compound, with respect to the subject method of treatment, refers to an amount of the therapeutic in a preparation which, when applied as part of a desired dosage regimen causes a increase in survival of a neuronal cell population according to clinically acceptable standards for the treatment or prophylaxis of a particular disorder.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term ‘IC₅₀’ refers to the concentration of an inhibitor composition that has 50% of the maximal inhibitory effect. Where the inhibitor composition inhibits cell growth, the IC₅₀ is the concentration that causes 50% of the maximal inhibition of cell growth.

The term “LD₅₀” means the dose of a drug that is lethal in 50% of test subjects.

A “patient” or “subject” to be treated by the subject method are mammals, including humans.

By “prevent degeneration” it is meant reduction in the loss of cells (such as from apoptosis), or reduction in impairment of cell function, e.g., release of dopamine in the case of dopaminergic neurons. Generally, as used herein, a therapeutic that “prevents” a disorder or condition refers to a compound that, in a sample, reduces the occurrence of the disorder or condition in the sample, relative to an untreated control sample, or delays the onset of one or more symptoms of the disorder or condition.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The term “therapeutic index” refers to the therapeutic index of a drug defined as LD₅₀/ED₅₀.

A “trophic factor” is a molecule that directly or indirectly affects the survival or function of a neuronal cell, e.g., a dopaminergic or GABAergic cell.

A “trophic amount” of a subject compound is an amount sufficient to, under the circumstances, cause an increase in the rate of survival or the functional performance of a neuronal cell, e.g., a dopaminergic or GABAergic cell.

‘Acyl’ refers to a group suitable for acylating a nitrogen atom to form an amide or carbamate, a carbon atom to form a ketone, a sulfur atom to form a thioester, or an oxygen atom to form an ester group, e.g., a hydrocarbon attached to a —C(═O)— moiety. Preferred acyl groups include benzoyl, acetyl, tert-butyl acetyl, pivaloyl, and trifluoroacetyl. More preferred acyl groups include acetyl and benzoyl. The most preferred acyl group is acetyl.

The term ‘acylamino’ is art-recognized and preferably refers to a moiety that can be represented by the general formula:

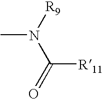

wherein R₉ and R′₁₁ each independently represent hydrogen or a hydrocarbon substituent, such as alkyl, heteroalkyl, aryl, heteroaryl, carbocyclic aliphatic, and heterocyclic aliphatic.

The terms ‘amine’ and ‘amino’ are art-recognized and refer to both unsubstituted and substituted amines as well as ammonium salts, e.g., as can be represented by the general formula:

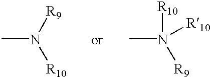

wherein R₉, R₁₀, and R′₁₀ each independently represent hydrogen or a hydrocarbon substituent, or R₉ and R₁₀ taken together with the N atom to which they are attached complete a heterocycle having from 4 to 8 atoms in the ring structure. In preferred embodiments, none of R₉, R₁₀, and R′₁₀ is acyl, e.g., R₉, R₁₀, and R′₁₀ are selected from hydrogen, alkyl, heteroalkyl, aryl, heteroaryl, carbocyclic aliphatic, and heterocyclic aliphatic. The term ‘alkylamine’ as used herein means an amine group, as defined above, having at least one substituted or unsubstituted alkyl attached thereto. Amino groups that are positively charged (e.g., R′₁₀ is present) are referred to as ‘ammonium’ groups. In amino groups other than ammonium groups, the amine is preferably basic, e.g., its conjugate acid has a pKₐ above 7.

The terms ‘amido’ and ‘amide’ are art-recognized as an amino-substituted carbonyl, such as a moiety that can be represented by the general formula:

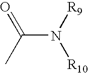

wherein R₉ and R₁₀ are as defined above. In certain embodiments, the amide will include imides.

‘Alkyl’ refers to a saturated or unsaturated hydrocarbon chain having 1 to 18 carbon atoms, preferably 1 to 12, more preferably 1 to 6, more preferably still 1 to 4 carbon atoms. Alkyl chains may be straight (e.g., n-butyl) or branched (e.g., sec-butyl, isobutyl, or t-butyl). Preferred branched alkyls have one or two branches, preferably one branch. Preferred alkyls are saturated. Unsaturated alkyls have one or more double bonds and/or one or more triple bonds. Preferred unsaturated alkyls have one or two double bonds or one triple bond, more preferably one double bond. Alkyl chains may be unsubstituted or substituted with from 1 to 4 substituents. Preferred alkyls are unsubstituted. Preferred substituted alkyls are mono-, di-, or trisubstituted. Preferred alkyl substituents include halo, haloalkyl, hydroxy, aryl (e.g., phenyl, tolyl, alkoxyphenyl, alkyloxycarbonylphenyl, halophenyl), heterocyclyl, and heteroaryl.

The terms ‘alkenyl’ and ‘alkynyl’ refer to unsaturated aliphatic groups analogous in length and possible substitution to the alkyls described above, but that contain at least one double or triple bond, respectively. When not otherwise indicated, the terms alkenyl and alkynyl preferably refer to lower alkenyl and lower alkynyl groups, respectively. When the term alkyl is present in a list with the terms alkenyl and alkynyl, the term alkyl refers to saturated alkyls exclusive of alkenyls and alkynyls.

The terms ‘alkoxyl’ and ‘alkoxy’ as used herein refer to an —O-alkyl group. Representative alkoxyl groups include methoxy, ethoxy, propyloxy, tert-butoxy, and the like. An ‘ether’ is two hydrocarbons covalently linked by an oxygen. Accordingly, the substituent of a hydrocarbon that renders that hydrocarbon an ether can be an alkoxyl, or another moiety such as —O-aryl, —O-heteroaryl, —O-heteroalkyl, —O-aralkyl, —O-heteroaralkyl, —O-carbocylic aliphatic, or —O-heterocyclic aliphatic.

The term ‘alkylthio’ refers to an —S-alkyl group. Representative alkylthio groups include methylthio, ethylthio, and the like. ‘Thioether’ refers to a sulfur atom bound to two hydrocarbon substituents, e.g., an ether wherein the oxygen is replaced by sulfur. Thus, a thioether substituent on a carbon atom refers to a hydrocarbon-substituted sulfur atom substituent, such as alkylthio or arylthio, etc.

The term ‘aralkyl’, as used herein, refers to an alkyl group substituted with an aryl group.

‘Aryl ring’ refers to an aromatic hydrocarbon ring system. Aromatic rings are monocyclic or fused bicyclic ring systems, such as phenyl, naphthyl, etc. Monocyclic aromatic rings contain from about 5 to about 10 carbon atoms, preferably from 5 to 7 carbon atoms, and most preferably from 5 to 6 carbon atoms in the ring. Bicyclic aromatic rings contain from 8 to 12 carbon atoms, preferably 9 or 10 carbon atoms in the ring. The term ‘aryl’ also includes bicyclic ring systems wherein only one of the rings is aromatic, e.g., the other ring is cycloalkyl, cycloalkenyl, or heterocyclyl. Aromatic rings may be unsubstituted or substituted with from 1 to about 5 substituents on the ring. Preferred aromatic ring substituents include: halo, cyano, lower alkyl, heteroalkyl, haloalkyl, phenyl, phenoxy, or any combination thereof. More preferred substituents include lower alkyl, cyano, halo, and haloalkyl.

‘Carbocyclic aliphatic ring’ refers to a saturated or unsaturated hydrocarbon ring. Carbocyclic aliphatic rings are not aromatic. Carbocyclic aliphatic rings are monocyclic, or are fused, spiro, or bridged bicyclic ring systems. Monocyclic carbocyclic aliphatic rings contain from about 4 to about 10 carbon atoms, preferably from 4 to 7 carbon atoms, and most preferably from 5 to 6 carbon atoms in the ring. Bicyclic carbocyclic aliphatic rings contain from 8 to 12 carbon atoms, preferably from 9 to 10 carbon atoms in the ring. Carbocyclic aliphatic rings may be unsubstituted or substituted with from 1 to 4 substituents on the ring. Preferred carbocyclic aliphatic ring substituents include halo, cyano, alkyl, heteroalkyl, haloalkyl, phenyl, phenoxy or any combination thereof. More preferred substituents include halo and haloalkyl. Preferred carbocyclic aliphatic rings include cyclopentyl, cyclohexyl, cyclohexenyl, cycloheptyl, and cyclooctyl. More preferred carbocyclic aliphatic rings include cyclohexyl, cycloheptyl, and cyclooctyl.

A ‘carbohydrate-modified polymer’ is a polymer that is covalently or associatively (i.e., through an inclusion complex) linked to one or more carbohydrate moieties.

The term ‘carbohydrate moiety’ is intended to include any molecule that is considered a carbohydrate by one of skill in the art and that is covalently bonded to a polymer. Carbohydrate moieties include mono- and polysaccharides. Carbohydrate moieties include trioses, tetroses, pentoses, hexoses, heptoses and monosaccharides of higher molecular weight (either D or L form), as well as polysaccharides comprising a single type of monosaccharide or a mixture of different monosaccharides. Polysaccharides may be of any polymeric conformation (e.g. branched, linear or circular). Examples of monosaccharides include glucose, fructose, and glucopyranose. Examples of polysaccharides include sucrose, lactose and cyclodextrin.

The term ‘carbonyl’ is art-recognized and includes such moieties as can be represented by the general formula:

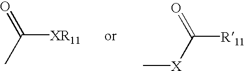

wherein X is a bond or represents an oxygen or a sulfur, and R₁₁ represents a hydrogen, hydrocarbon substituent, or a pharmaceutically acceptable salt, R_{11′} represents a hydrogen or hydrocarbon substituent. Where X is an oxygen and R₁₁ or R_{11′} is not hydrogen, the formula represents an ‘ester’. Where X is an oxygen, and R₁₁ is as defined above, the moiety is referred to herein as a carboxyl group, and particularly when R₁₁ is a hydrogen, the formula represents a ‘carboxylic acid’. Where X is an oxygen, and R_{11′} is hydrogen, the formula represents a ‘formate’. In general, where the oxygen atom of the above formula is replaced by sulfur, the formula represents a ‘thiocarbonyl’ group. Where X is a sulfur and R₁₁ or R_{11′} is not hydrogen, the formula represents a ‘thioester.’ Where X is a sulfur and R₁₁ is hydrogen, the formula represents a ‘thiocarboxylic acid.’ Where X is a sulfur and R_{11′}0 is hydrogen, the formula represents a ‘thioformate.’ On the other hand, where X is a bond, R₁₁ is not hydrogen, and the carbonyl is bound to a hydrocarbon, the above formula represents a ‘ketone’ group. Where X is a bond, R₁₁ is hydrogen, and the carbonyl is bound to a hydrocarbon, the above formula represents an ‘aldehyde’ or ‘formyl’ group.

‘Ci alkyl’ is an alkyl chain having i member atoms. For example, C4 alkyls contain four carbon member atoms. C4 alkyls containing may be saturated or unsaturated with one or two double bonds (cis or trans) or one triple bond. Preferred C4 alkyls are saturated. Preferred unsaturated C4 alkyl have one double bond. C4 alkyl may be unsubstituted or substituted with one or two substituents. Preferred substituents include lower alkyl, lower heteroalkyl, cyano, halo, and haloalkyl.

‘Halogen’ refers to fluoro, chloro, bromo, or iodo substituents. Preferred halo are fluoro, chloro and bromo; more preferred are chloro and fluoro.

‘Haloalkyl’ refers to a straight, branched, or cyclic hydrocarbon substituted with one or more halo substituents. Preferred haloalkyl are C1-C12; more preferred are C1-C6; more preferred still are C1-C3. Preferred halo substituents are fluoro and chloro. The most preferred haloalkyl is trifluoromethyl.

‘Heteroalkyl’ is a saturated or unsaturated chain of carbon atoms and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl have one or two branches, preferably one branch. Preferred heteroalkyl are saturated. Unsaturated heteroalkyl have one or more double bonds and/or one or more triple bonds. Preferred unsaturated heteroalkyl have one or two double bonds or one triple bond, more preferably one double bond. Heteroalkyl chains may be unsubstituted or substituted with from 1 to about 4 substituents unless otherwise specified. Preferred heteroalkyl are unsubstituted. Preferred heteroalkyl substituents include halo, aryl (e.g., phenyl, tolyl, alkoxyphenyl, alkoxycarbonylphenyl, halophenyl), heterocyclyl, heteroaryl. For example, alkyl chains substituted with the following substituents are heteroalkyl: alkoxy (e.g., methoxy, ethoxy, propoxy, butoxy, pentoxy), aryloxy (e.g., phenoxy, chlorophenoxy, tolyloxy, methoxyphenoxy, benzyloxy, alkoxycarbonylphenoxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkoxycarbonylphenylthio), amino (e.g., amino, mono- and di-C1-C3 alkylamino, methylphenylamino, methylbenzylamino, C1-C3 alkylamido, carbamamido, ureido, guanidino).

‘Heteroatom’ refers to a multivalent non-carbon atom, such as a boron, phosphorous, silicon, nitrogen, sulfur, or oxygen atom, preferably a nitrogen, sulfur, or oxygen atom. Groups containing more than one heteroatom may contain different heteroatoms.

‘Heteroaryl ring’ refers to an aromatic ring system containing carbon and from 1 to about 4 heteroatoms in the ring. Heteroaromatic rings are monocyclic or fused bicyclic ring systems. Monocyclic heteroaromatic rings contain from about 5 to about 10 member atoms (carbon and heteroatoms), preferably from 5 to 7, and most preferably from 5 to 6 in the ring. Bicyclic heteroaromatic rings contain from 8 to 12 member atoms, preferably 9 or 10 member atoms in the ring. The term ‘heteroaryl’ also includes bicyclic ring systems wherein only one of the rings is aromatic, e.g., the other ring is cycloalkyl, cycloalkenyl, or heterocyclyl. Heteroaromatic rings may be unsubstituted or substituted with from 1 to about 4 substituents on the ring. Preferred heteroaromatic ring substituents include halo, cyano, lower alkyl, heteroalkyl, haloalkyl, phenyl, phenoxy or any combination thereof. Preferred heteroaromatic rings include thienyl, thiazolyl, oxazolyl, pyrrolyl, purinyl, pyrimidyl, pyridyl, and furanyl. More preferred heteroaromatic rings include thienyl, furanyl, and pyridyl.

‘Heterocyclic aliphatic ring’ is a non-aromatic saturated or unsaturated ring containing carbon and from 1 to about 4 heteroatoms in the ring, wherein no two heteroatoms are adjacent in the ring and preferably no carbon in the ring attached to a heteroatom also has a hydroxyl, amino, or thiol group attached to it. Heterocyclic aliphatic rings are monocyclic, or are fused or bridged bicyclic ring systems. Monocyclic heterocyclic aliphatic rings contain from about 4 to about 10 member atoms (carbon and heteroatoms), preferably from 4 to 7, and most preferably from 5 to 6 member atoms in the ring. Bicyclic heterocyclic aliphatic rings contain from 8 to 12 member atoms, preferably 9 or 10 member atoms in the ring. Heterocyclic aliphatic rings may be unsubstituted or substituted with from 1 to about 4 substituents on the ring. Preferred heterocyclic aliphatic ring substituents include halo, cyano, lower alkyl, heteroalkyl, haloalkyl, phenyl, phenoxy or any combination thereof. More preferred substituents include halo and haloalkyl. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, hydantoin, oxazoline, imidazolinetrione, triazolinone, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Preferred heterocyclic aliphatic rings include piperazyl, morpholinyl, tetrahydrofuranyl, tetrahydropyranyl and piperidyl. Heterocycles can also be polycycles.

The term ‘hydroxyl’ means —OH.

‘Lower alkyl’ refers to an alkyl chain comprised of 1 to 5, preferably 1 to 4 carbon member atoms, more preferably 1 or 2 carbon member atoms. Lower alkyls may be saturated or unsaturated. Preferred lower alkyls are saturated. Lower alkyls may be unsubstituted or substituted with one or about two substituents. Preferred substituents on lower alkyl include cyano, halo, trifluoromethyl, amino, and hydroxyl. Throughout the application, preferred alkyl groups are lower alkyls. In preferred embodiments, a substituent designated herein as alkyl is a lower alkyl. Likewise, ‘lower alkenyl’ and ‘lower alkynyl’ have similar chain lengths.

‘Lower heteroalkyl’ refers to a heteroalkyl chain comprised of 1 to 4, preferably 1 to 3 member atoms, more preferably 1 to 2 member atoms. Lower heteroalkyl contain one or two non-adjacent heteroatom member atoms. Preferred lower heteroalkyl contain one heteroatom member atom. Lower heteroalkyl may be saturated or unsaturated. Preferred lower heteroalkyl are saturated. Lower heteroalkyl may be unsubstituted or substituted with one or about two substituents. Preferred substituents on lower heteroalkyl include cyano, halo, trifluoromethyl, and hydroxyl.

‘Mi heteroalkyl’ is a heteroalkyl chain having i member atoms. For example, M4 heteroalkyls contain one or two non-adjacent heteroatom member atoms. M4 heteroalkyls containing 1 heteroatom member atom may be saturated or unsaturated with one double bond (cis or trans) or one triple bond. Preferred M4 heteroalkyl containing 2 heteroatom member atoms are saturated. Preferred unsaturated M4 heteroalkyl have one double bond. M4 heteroalkyl may be unsubstituted or substituted with one or two substituents. Preferred substituents include lower alkyl, lower heteroalkyl, cyano, halo, and haloalkyl.

‘Member atom’ refers to a polyvalent atom (e.g., C, O, N, or S atom) in a chain or ring system that constitutes a part of the chain or ring. For example, in cresol, six carbon atoms are member atoms of the ring and the oxygen atom and the carbon atom of the methyl substituent are not member atoms of the ring.

As used herein, the term ‘nitro’ means —NO₂.

‘Pharmaceutically acceptable salt’ refers to a cationic salt formed at any acidic (e.g., hydroxamic or carboxylic acid) group, or an anionic salt formed at any basic (e.g., amino or guanidino) group. Such salts are well known in the art. See e.g., World Patent Publication 87/05297, Johnston et al., published Sep. 11, 1987, incorporated herein by reference. Such salts are made by methods known to one of ordinary skill in the art. It is recognized that the skilled artisan may prefer one salt over another for improved solubility, stability, formulation ease, price and the like. Determination and optimization of such salts is within the purview of the skilled artisan's practice. Preferred cations include the alkali metals (such as sodium and potassium), and alkaline earth metals (such as magnesium and calcium) and organic cations, such as trimethylammonium, tetrabutylammonium, etc. Preferred anions include halides (such as chloride), sulfonates, carboxylates, phosphates, and the like. Clearly contemplated in such salts are addition salts that may provide an optical center where once there was none. For example, a chiral tartrate salt may be prepared from the compounds of the invention. This definition includes such chiral salts.

‘Phenyl’ is a six-membered monocyclic aromatic ring that may or may not be substituted with from 1 to 5 substituents. The substituents may be located at the ortho, meta or para position on the phenyl ring, or any combination thereof. Preferred phenyl substituents include: halo, cyano, lower alkyl, heteroalkyl, haloalkyl, phenyl, phenoxy or any combination thereof. More preferred substituents on the phenyl ring include halo and haloalkyl. The most preferred substituent is halo.

The terms ‘polycyclyl’ and ‘polycyclic group’ refer to two or more rings (e.g., cycloalkyls, cycloalkenyls, heteroaryls, aryls and/or heterocyclyls) in which two or more member atoms of one ring are member atoms of a second ring. Rings that are joined through non-adjacent atoms are termed ‘bridged’ rings, and rings that are joined through adjacent atoms are ‘fused rings’.

The term ‘sulthydryl’ means —SH, and the term ‘sulfonyl’ means —SO₂—.

A ‘substitution’ or ‘substituent’ on a small organic molecule generally refers to a position on a multi-valent atom bound to a moiety other than hydrogen, e.g., a position on a chain or ring exclusive of the member atoms of the chain or ring. Such moieties include those defined herein and others as are known in the art, for example, halogen, alkyl, alkenyl, alkynyl, azide, haloalkyl, hydroxyl, carbonyl (such as carboxyl, alkoxycarbonyl, formyl, ketone, or acyl), thiocarbonyl (such as thioester, thioacetate, or thioformate), alkoxyl, phosphoryl, phosphonate, phosphinate, amine, amide, amidine, imine, cyano, nitro, azido, sulthydryl, alkylthio, sulfate, sulfonate, sulfamoyl, sulfonamido, sulfonyl, silyl, ether, cycloalkyl, heterocyclyl, heteroalkyl, heteroalkenyl, and heteroalkynyl, heteroaralkyl, aralkyl, aryl or heteroaryl. It will be understood by those skilled in the art that certain substituents, such as aryl, heteroaryl, polycyclyl, alkoxy, alkylamino, alkyl, cycloalkyl, heterocyclyl, alkenyl, alkynyl, heteroalkyl, heteroalkenyl, and heteroalkynyl, can themselves be substituted, if appropriate. This invention is not intended to be limited in any manner by the permissible substituents of organic compounds. It will be understood that ‘substitution’ or ‘substituted with’ includes the implicit proviso that such substitution is in accordance with permitted valence of the substituted atom and the substituent, and that the substitution results in a stable compound, e.g., which does not spontaneously undergo transformation such as by rearrangement, cyclization, elimination, hydrolysis, etc.

As used herein, the definition of each expression, e.g., alkyl, m, n, etc., when it occurs more than once in any structure, is intended to be independent of its definition elsewhere in the same structure.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl, and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The terms ortho, meta and para apply to 1,2-, 1,3- and 1,4-disubstituted benzenes, respectively. For example, the names 1,2-dimethylbenzene and ortho-dimethylbenzene are synonymous.

The phrase ‘protecting group’ as used herein means temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991; and Kocienski, P. J. Protecting Groups, Georg Thieme Verlag: New York, 1994).

For purposes of this invention, the chemical elements are identified in accordance with the Periodic Table of the Elements, CAS version, Handbook of Chemistry and Physics, 67th Ed., 1986-87, inside cover. Also for purposes of this invention, the term ‘hydrocarbon’ is contemplated to include all permissible compounds or moieties having at least one carbon-hydrogen bond. In a broad aspect, the permissible hydrocarbons include acyclic and cyclic, branched and unbranched, carbocyclic and heterocyclic, aromatic and nonaromatic organic compounds which can be substituted or unsubstituted.

Contemplated equivalents of the compounds described above include compounds which otherwise correspond thereto, and which have the same useful properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

III. Exemplary Polymer Compositions

The subject polymers include linear and/or branched poly(ethylenimine) polymers that have been modified by attaching carbohydrate moieties, such as cyclodextrin, to the polymer backbone (e.g., through attachment to nitrogen atoms in the polymer chain). The polymers (prior to carbohydrate modification) preferably have molecular weights of at least 2,000, such as 2,000 to 100,000, preferably 5,000 to 80,000. In certain embodiments, the subject polymers have a structure of the formula:

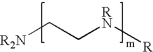

wherein R represents, independently for each occurrence, H, lower alkyl, a carbohydrate moiety (optionally attached via a linker moiety, such as an alkylene chain or a polyethylene glycol oligomer), or

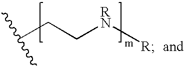

m, independently for each occurrence, represents an integer greater than 10, e.g., from 10-10,000, preferably from 10 to 5,000, or from 100 to 1,000.

In certain embodiments, R includes a carbohydrate moiety for at least about 1%, more preferably at least about 2%, or at least about 3%, and up to about 5% or even 10%, 15%, or 20% of its occurrences.

In certain embodiments, the polymer is linear, i.e., no occurrence of R represents

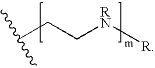

In certain embodiments, the carbohydrate moieties make up at least about 2%, 3% or 4% by weight, up to 5%, 7%, or even 10% of the carbohydrate-modified polymer by weight. Where the carbohydrate moieties include cyclodextrin, carbohydrate moieties may be 2% of the weight of the copolymer, preferably at least 5% or 10%, or even as much as 20%, 40%, 50%, 60%, 80%, or even 90% of the weight of the copolymer.

In certain embodiments, at least about 2%, 3% or 4%, up to 5%, 7%, or even 10%, 15%, 20%, or 25% of the ethylenimine subunits in the polymer are modified with a carbohydrate moiety. In certain such embodiments, however, no more than 25%, 30%, 35%, 40%, or 50% of the ethylenimine subunits are so modified. In preferred embodiments, the level of carbohydrate modification is selected such that the toxicity is less than 20% of the toxicity of the unmodified polymer, yet the transfection efficiency is at least 30% of the efficiency of the corresponding polymer modified at 5% of the ethylenimine subunits. Preferably, one out of every 6 to 15 ethylenimine subunits is modified with a carbohydrate moiety.

Copolymers of poly(ethylenimine) that bear nucleophilic amino substituents susceptible to derivatization with cyclodextrin moieties can also be used to prepare cyclodextrin-modified polymers within the scope of the present invention. Exemplary extents of carbohydrate modification are 10-15% of the ethyleneimine moieties, 15-20% of the ethylenimine moieties, 20-25% of the ethylenimine moieties, 25-30% of the ethylenimine moieties, 30-40% of the ethylenimine moieties, or a combination of two or more of these ranges.

Where the carbohydrate moiety is attached through a linker, the linker group(s) may be an alkylene chain, a polyethylene glycol (PEG) chain, polysuccinic anhydride, polysebacic acid (PSA), poly-L-glutamic acid, poly(ethyleneimine), an oligosaccharide, an amino acid chain, or any other suitable linkage. More than one type of linker may be present in a given polymer or polymerization reaction. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo- or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties (e.g., the polymer chain and the carbohydrate) by any suitable bond or functional group, including carbon-carbon bonds, esters, ethers, amides, amines, carbonates, carbamates, ureas, sulfonamides, etc.

In certain embodiments the linker group(s) of the present invention represent a hydrocarbylene group wherein one or more methylene groups is optionally replaced by a group Y (provided that none of the Y groups are adjacent to each other), wherein each Y, independently for each occurrence, is selected from, substituted or unsubstituted aryl, heteroaryl, cycloalkyl, heterocycloalky, or —O—, C(═X) (wherein X is NR₁, O or S), —OC(O)—, —C(═O)O, —NR₁—, —NR₁CO—, —C(O)NR₁—, —S(O)ₙ— (wherein n is 0, 1, or 2), —OC(O)—NR₁, —NR₁—C(O)—NR₁—, —NR₁—C(═NR₁)—NR₁—, and —B(OR₁)—; and R₁, independently for each occurrence, represents H or a lower alkyl.

In certain embodiments the linker group represents a derivatized or non-derivatized amino acid. In certain embodiments linking groups with one or more terminal carboxyl groups may be conjugated to the polymer. In certain embodiments, one or more of these terminal carboxyl groups may be capped by covalently attaching them to a therapeutic agent or a cyclodextrin moiety via an (thio)ester or amide bond. In still other embodiments linking groups with one or more terminal hydroxyl, thiol, or amino groups may be incorporated into the polymer. In preferred embodiments, one or more of these terminal hydroxyl groups may be capped by covalently attaching them to a therapeutic agents or a carbohydrate (e.g., cyclodextrin) moiety via a carbonate, carbamate, thiocarbonate, or thiocarbamate bond. In certain embodiments, these (thio)ester, amide, (thio)carbonate or (thio)carbamate bonds may be biohydrolyzable, i.e., capable of being hydrolyzed under biological conditions.

In certain embodiments, carbohydrate moieties can be attached to the polymer via a non-covalent associative interaction. For example, the polymer chain can be modified with groups, such as adamantyl groups, that form inclusion complexes with cyclodextrin. The modified polymer can then be combined with compound that includes a cyclodextrin moiety and, optionally, a carbohydrate moiety (which may be a second cyclodextrin moiety, e.g., the compound may be symmetrical) under conditions suitable for forming inclusion complexes between the polymer and the compound, resulting in a complex such as polymer-adamantane::cyclodextrin-linker-carbohydrate. In this way, a polymer can be modified with carbohydrates without covalently attaching carbohydrates to the polymer itself. Similarly, a cyclodextrin-modified polymer as described herein can be treated with molecule having polyethylene glycol (PEG) chains linked to groups that form inclusion complexes with cyclodextrin. As described in greater detail below, particles of polymers modified in this way are stabilized (e.g., due to the presence of a PEG “brush layer” on their surface) relative to particles in which no such inclusion complexes have been formed. Alternatively or additionally, inclusion complexes can be used to couple ligands to the polymer (e.g., for targeting the polymer to a particular tissue, organ, or other region of a patient's body), or to otherwise modify the physical, chemical, or biological properties of the polymer.

Exemplary cyclodextrin moieties include cyclic structures consisting essentially of from 6 to 8 saccharide moieties, such as cyclodextrin and oxidized cyclodextrin. A cyclodextrin moiety optionally comprises a linker moiety that forms a covalent linkage between the cyclic structure and the polymer backbone, preferably having from 1 to 20 atoms in the chain, such as alkyl chains, including dicarboxylic acid derivatives (such as glutaric acid derivatives, succinic acid derivatives, and the like), and heteroalkyl chains, such as oligoethylene glycol chains. Cyclodextrin moieties may further include one or more carbohydrate moieties, preferably simple carbohydrate moieties such as galactose, attached to the cyclic core, either directly (i.e., via a carbohydrate linkage) or through a linker group.

Cyclodextrins are cyclic polysaccharides containing naturally occurring D-(+)-glucopyranose units in an α-(1,4) linkage. The most common cyclodextrins are alpha ((α)-cyclodextrins, beta (β)-cyclodextrins and gamma (γ)-cyclodextrins which contain, respectively. six, seven, or eight glucopyranose units. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. Thus, using (β)-cyclodextrin as an example, a cyclodextrin is often represented schematically as follows.

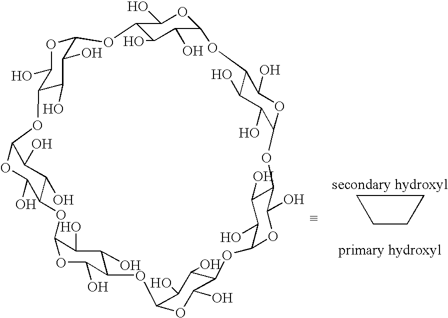

The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); T. Cserhati, Analytical Biochemistry, 225:328-332(1995); Husain et al., Applied Spectroscopy, 46:652-658 (1992); FR 2 665 169). Additional methods for modifying polymers are disclosed in Suh, J. and Noh, Y., Bioorg. Med. Chem. Lett. 1998, 8, 1327-1330.

Cyclodextrins have been used as a delivery vehicle of various therapeutic compounds by forming inclusion complexes with various drugs that can fit into the hydrophobic cavity of the cyclodextrin or by forming non-covalent association complexes with other biologically active molecules such as oligonucleotides and derivatives thereof. For example, see U.S. Pat. Nos. 4,727,064, 5,608,015, 5,276,088, and 5,691,316. Various cyclodextrin-containing polymers and methods of their preparation are also known in the art. Comprehensive Supramolecular Chemistry, Volume 3, J. L. Atwood et al., eds., Pergamon Press (1996).

IV Exemplary Applications of Method and Compositions

Therapeutic compositions according to the invention contain a therapeutic agent and a carbohydrate-modified polymer of the invention, such as, for example, a cyclodextrin-modified polymer of the invention or a carbohydrate-modified polymer having an IC₅₀ for cells in culture of greater than 25 μg/ml. The therapeutic agent may be any synthetic or naturally occurring biologically active therapeutic agent including those known in the art. Examples of suitable therapeutic agents include, but are not limited to, antibiotics, steroids, polynucleotides (e.g., genomic DNA, cDNA, mRNA and antisense oligonucleotides), plasmids, peptides, peptide fragments, small molecules (e.g., doxorubicin) and other biologically active macromolecules such as, for example, proteins and enzymes. Therapeutic compositions are preferably sterile and/or non-pyrogenic, e.g., do not substantially raise a patient's body temperature after administration.

A therapeutic composition of the invention may be prepared by means known in the art. In a preferred embodiment, a copolymer of the invention is mixed with a therapeutic agent, as described above, and allowed to self-assemble. According to the invention, the therapeutic agent and a carbohydrate-modified polymer of the invention associate with one another such that the copolymer acts as a delivery vehicle for the therapeutic agent. The therapeutic agent and carbohydrate-modified polymer may associate by means recognized by those of skill in the art such as, for example, electrostatic interaction and hydrophobic interaction. The degree of association may be determined by techniques known in the art including, for example, fluorescence studies, DNA mobility studies, light scattering, electron microscopy, and will vary depending upon the therapeutic agent. As a mode of delivery, for example, a therapeutic composition of the invention containing a copolymer of the invention and DNA may be used to aid in transfection, i.e., the uptake of DNA into an animal (e.g., human) cell. (Boussif, O. Proceedings of the National Academy of Sciences, 92:7297-7301(1995); Zanta et al. Bioconjugate Chemistry, 8:839-844 (1997)).

A therapeutic composition of the invention may be, for example, a solid, liquid, suspension, or emulsion. Preferably a therapeutic composition of the invention is in a form that can be injected, e.g., intratumorally or intravenously. Other modes of administration of a therapeutic composition of the invention include, depending on the state of the therapeutic composition, methods known in the art such as, but not limited to, oral administration, topical application, parenteral, intravenous, intranasal, intraocular, intracranial or intraperitoneal injection.

Depending upon the type of therapeutic agent used, a therapeutic composition of the invention may be used in a variety of therapeutic methods (e.g. DNA vaccines, antibiotics, antiviral agents) for the treatment of inherited or acquired disorders such as, for example, cystic fibrosis, Gaucher's disease, muscular dystrophy, AIDS, cancers (e.g., multiple myeloma, leukemia, melanoma, and ovarian carcinoma), cardiovascular conditions (e.g., progressive heart failure, restenosis, and hemophilia), and neurological conditions (e.g., brain trauma).

In certain embodiments according to the invention, a method of treatment administers a therapeutically effective amount of a therapeutic composition of the invention. A therapeutically effective amount, as recognized by those of skill in the art, will be determined on a case by case basis. Factors to be considered include, but are not limited to, the disorder to be treated and the physical characteristics of the one suffering from the disorder.

Another embodiment of the invention is a composition containing at least one biologically active compound having agricultural utility and a linear cyclodextrin-modified polymer or a linear oxidized cyclodextrin-modified polymer of the invention. The agriculturally biologically active compounds include those known in the art. For example, suitable agriculturally biologically active compounds include, but are not limited to, fungicides, herbicides, insecticides, and mildewcides.

Exemplification

The invention now being generally described, it will be more readily understood by reference to the following examples, which are included merely for purposes of illustration of certain aspects and embodiments of the present invention, and are not intended to limit the invention.

### EXAMPLE 1

Synthesis and Characterization of CD-bPEI With Altered CD Loading

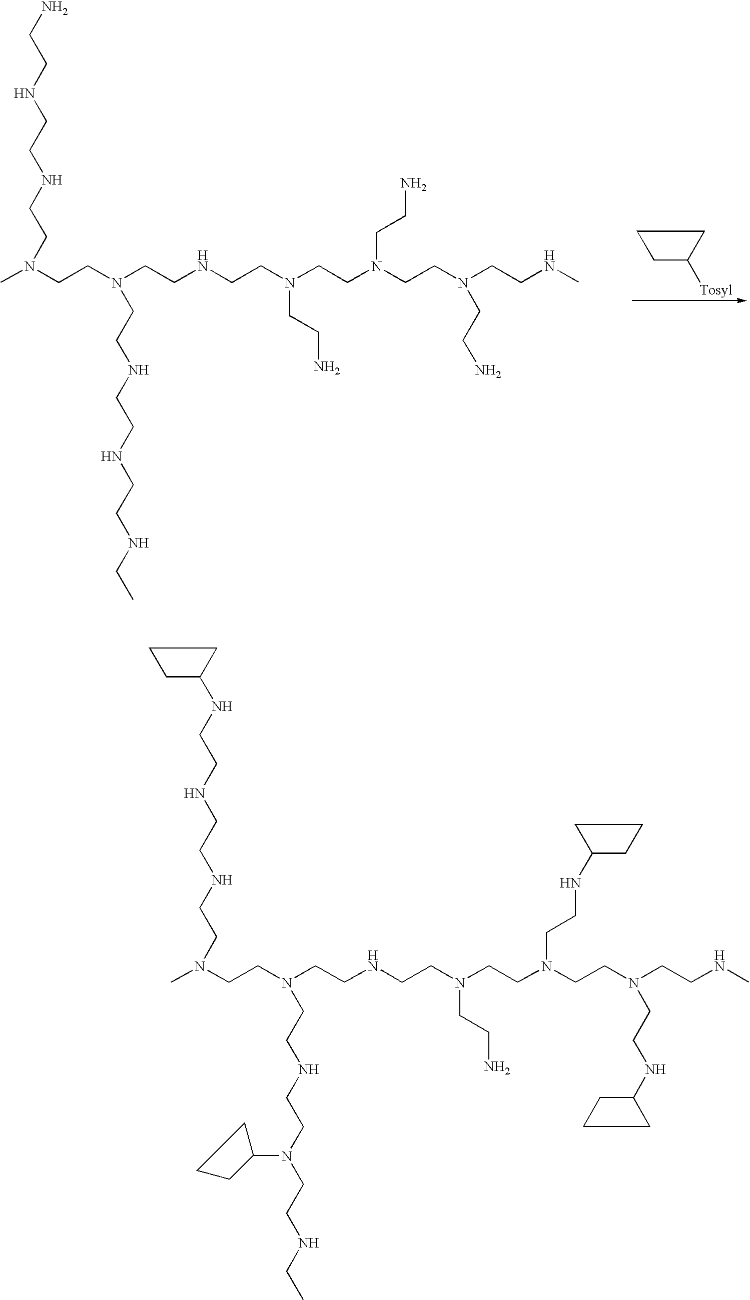

Branched PEI_{25,000} (295.6 mg, Aldrich) and 6-monotosyl-β-cyclodextrin (2.287 g, Cyclodextrin Technologies Development, Inc.) were dissolved in 100 mL of various H₂O/DMSO solvent mixture (Table 1). The resulting mixture was stirred at 70° C. for 72 h. The solution turned slightly yellow. The solution was then transferred to a Spectra/Por MWCO 10,000 membrane and dialyzed against water for 6 days. Water was then removed by lyophilization to afford a slightly colored solid. Cyclodextrin/PEI ratio was calculated based on the proton integration of ¹H NMR (Varian 300 Hz, D₂O) δ 5.08 ppm (s br., C₁H of CD), 3.3-4.1 ppm (m br. C₂H-C₆H of CD), 2.5-3.2 ppm (m br. CH₂ of PEI).

The cyclodextrin loading on PEI was found to increase with decreasing amounts of H₂O in the reaction mixture (Table 1).

TABLE 1
Effect of H₂O on cyclodextrin loading

| H₂O/DMSO | Amount of water | |
|---|---|---|
| (mL) | (%) | Ethyleneimine/CD |
| 60/40 | 60 | 19.9 |
| 40/60 | 40 | 16.8 |
| 20/80 | 20 | 14.7 |
| 5/95 | 5 | 12.6 |
| 1/99 | 1 | 10.5 |
| 0.1/99.9 | 0.1 | 8.4 |
| 0/100 | 0 | 6.3 |

### EXAMPLE 2

Synthesis of Linear PEI-CD

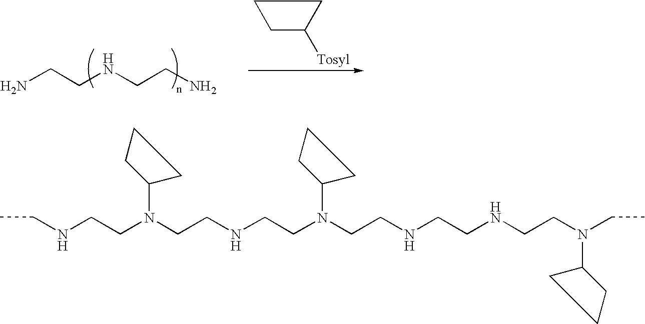

Low loading: Linear PEI (50 mg, Polysciences, Inc., MW 25,000) was dissolved in dry DMSO (5 mL). Cyclodextrin monotosylate (189 mg, 75 eq., Cyclodextrin Technologies Development, Inc.) was added to the solution. The solution was stirred under Argon at 70-72° C. for 4 days. Then this solution was dialyzed in water (total dialysis volume around 50 mL) for six days (Spectra/Por 7 MWCO 25,000 membrane). 1PEI-CD (46 mg) was obtained after lyophilization. ¹H NMR (Bruker AMX 500 MHz, D₂O) δ 5.09 (s br., C1 of CD), 3.58-4.00 (m br., C2-C6 of CD), 2.98 (m br., PEI). 8.8% of PEI repeats were conjugated with CD.

High loading: Linear PEI (50 mg, Polysciences, Inc. MW 25,000) was dissolved in dry DMSO (10 mL). Cyclodextrin monotosylate (773 mg, 300 eq., Cyclodextrin Technologies Development, Inc.) was added to the solution. The solution was stirred under argon at 70-72° C. for 4 days. Then this solution was dialyzed in water (total dialysis volume around 50 mL) for six days (Spectra/Por 7 MWCO 25,000 membrane). Precipitation in dialysis bag was observed. The precipitate (unreacted CD-monotosylate) was removed using 0.2 μM syringe filter and the filtrant was dialyzed in a 25,000 MWCO membrane for another 24 hours. 1PEI-CD (75 mg) was obtained after lyophilization. ¹H NMR (Bruker AMX 500 MHz, D₂O) δ 5.09 (s br., C1 of CD), 3.58-4.00 (m br., C2-C6 of CD), 2.98 (m br., PEI). 11.6% of PEI repeats were conjugated with CD.

### EXAMPLE 3

Synthesis and Characterization of CD-1PEI With Altered CD Loading

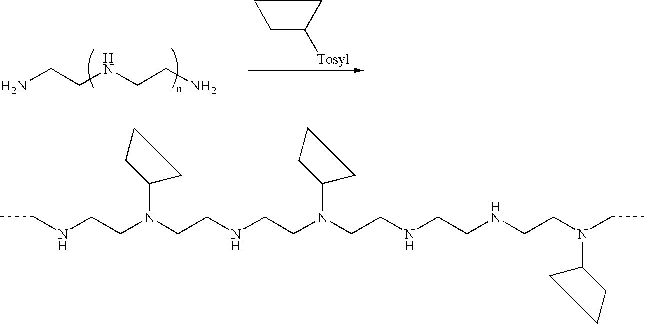

Linear PEI_{25,000} (500 mg, Polysciences, Inc.) and 6-monotosyl-β-cyclodextrin (3.868 g, Cyclodextrin Technologies Development, Inc.) were dissolved in 36 mL of DMSO. The resulting mixture was stirred at 70° C. for 6 days. The solution turned slightly yellow. The solution was then transferred to a Spectra/Por MWCO 10,000 membrane and dialyzed against water for 6 days. Water was then removed by lyophilization to afford a slightly colored solid. Cyclodextrin/PEI ratio was calculated based on the proton integration of ¹H NMR (Varian 300 MHz, D₂O) δ 5.08 ppm (s br., C₁H of CD), 3.3-4.1 ppm (m br. C₂H-C₆H of CD), 2.5-3.2 ppm (m br. CH₂ of PEI). In this example, the cyclodextrin/PEI ratio was 8.4.

### EXAMPLE 4

Formulations of CD-PEI With Plasmids: Salt Stabilization With AD-PEG Material

Plasmid DNA (pGL3-CV, plasmid containing the luciferase gene under the control of an SV40 promoter) was prepared at 0.5 mg/mL in water. Branched CD-PEI was prepared at 2.0 mg/mL in water. AD-PEG₅₀₀₀ was prepared at 10 mg/mL and 100 mg/mL in water. (See Examples 22-28 of U.S. patent application Ser. No. 10/021,312, filed Dec. 19, 2001, for details.)

Polyplexes were prepared by mixing the desired amount of AD-PEG₅₀₀₀ with 6 μL of branched CD-PEI. This polymer solution was then added to 6 μL of DNA solution.

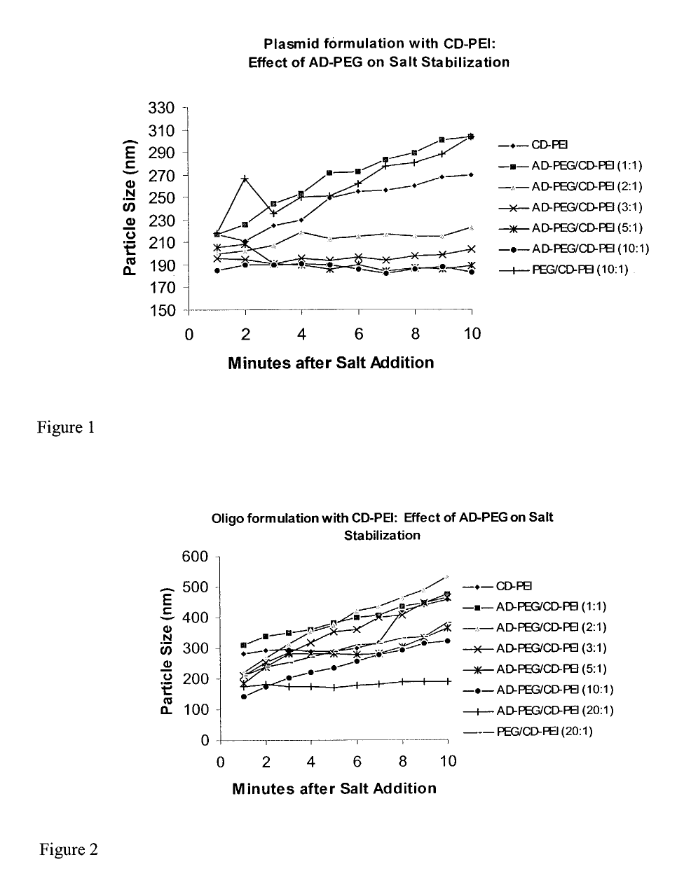

Polyplex solutions were transferred to a light-scattering cuvette. 1.6 mL of PBS (150 mM) was added and particle size measured immediately following salt addition for 10 minutes using a Zeta Pals dynamic light scattering detector (Brookhaven Instruments). Results are depicted in FIG. 1.

Formulations of CD-PEI With Oligos: Salt Stabilization With AD-PEG

Oligo DNA (FITC-Oligo) was prepared at 0.5 mg/mL in water. Branched CD-PEI was prepared at 2.0 mg/mL in water. AD-PEG₅₀₀₀ was prepared at 10 mg/mL and 100 mg/mL in water.

Polyplexes were prepared by mixing the desired amount of AD-PEG₅₀₀₀ with 6 μL of branched CD-PEI. This polymer solution was then added to 6 μL of DNA solution.

Polyplex solutions were transferred to a light-scattering cuvette. 1.6 mL of PBS (150 mM) was added and particle size measured immediately following salt addition for 10 minutes using a Zeta Pals dynamic light scattering detector (Brookhaven Instruments). Results are depicted in FIG. 2.

### EXAMPLE 5

Plasmid Transfection in vitro

PC3 cells were plated at 200,000 cells/mL in 24-well plates. After 24 hours, the cells were transfected with 3 μg/well of pEGFP-Luc (plasmid containing the EGFP-Luc fusion gene under the control of a CMV promoter) complexed with branched CD-PEI at a 5:1 weight ratio. (For each well, transfection mixtures were prepared in 60 μL of water and then 1 mL of OptiMEM (a serum-free medium from Life Technologies) was added to the solutions. The final solutions were then transferred to the cells.) 4 hours after transfection, media was removed and replaced with 5 mL of complete media. Cells were analyzed by flow cytometry for EGFP expression 48 hours after transfection. EGFP expression was observed in 25% of analyzed cells.

Oligo Delivery by Branched CD-PEI

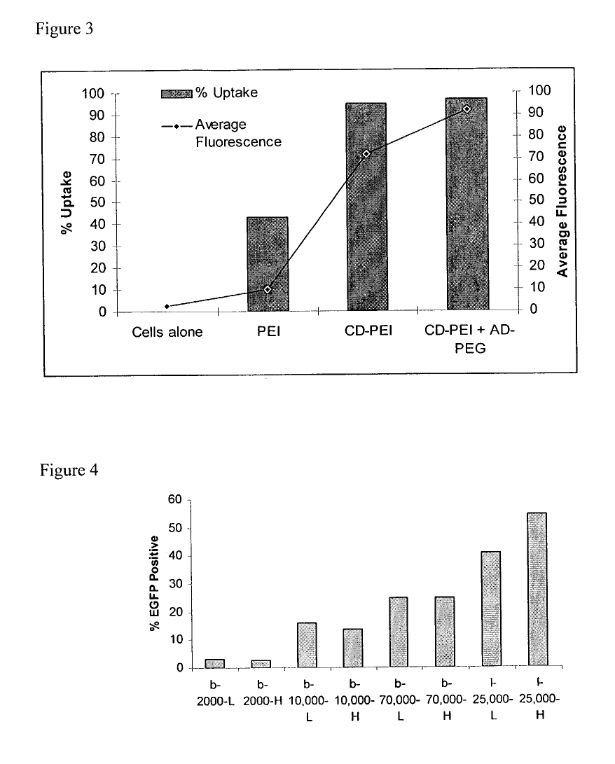

PC3 cells were plated at 300,000 cells/well in 6-well plates. After 24 hours, the cells were transfected with 3 μg/well of FITC-Oligo complexed with branched PEI (modified and unmodified) or branched CD-PEI at a 5:1 weight ratio. 15 minutes after transfection, cells were washed with PBS, trypsinized and analyzed by flow cytometry for uptake of the fluorescent oligos. EGFP expression was observed in 25% of analyzed cells. Results are depicted in FIG. 3.

Transfection Efficiencies of Various CD-PEI Polymers

PC3 cells were transfected with several CD-PEI polymers as listed below.

| Polymer | Mass/monomer | ethylenimine/CD |
|---|---|---|
| b-PEI2000-CD-L | 178 | 9.5 |
| b-PEI2000-CD-H | 216 | 7.4 |
| b-PEI10000-CD-L | 89 | 27 |
| b-PEI10000-CD-H | 111 | 19 |
| b-PEI70000-CD-L | 98 | 23 |
| b-PEI70000-CD-H | 119 | 16.8 |
| l-PEI25000-CD-L | 155 | 11.4 |
| l-PEI25000-CD-H | 192 | 8.6 |

The nomenclature is defined as follows: b-PEI2000-CD-L is cyclodextrin grafted to branched PEI of 2000 MW. A prefix of ‘1’ indicates a linear PEI substrate. The “L” and “H” stands for “lighter” and “heavier” grafted polymers (see the respective ethylenimine/CD ratios as listed on the right-most column). The CD-PEI polymers were prepared according to the protocol described in Example 1.

PC3 cells were plated at 200,000 cells/well in 6-well plates. After 24 hours, the cells were transfected with 3 μg of plasmid of pEGFP-Luc plasmid assembled with CD-PEI polymers at 15 N/P in 1 mL of Optimem. Five hours after transfection, 4 mL of complete media was added to each well. Cells were trypsinized, collected, and analyzed by flow cytometry for EGFP expression 48 hours after transfection. The results are shown in FIG. 4. High transfection efficiency was observed with increasing molecular weight. Linear-PEI-based conjugates transfected with higher efficiency than branched-PEI-based conjugates.

### EXAMPLE 6

Toxicity of CD-PEI in vitro

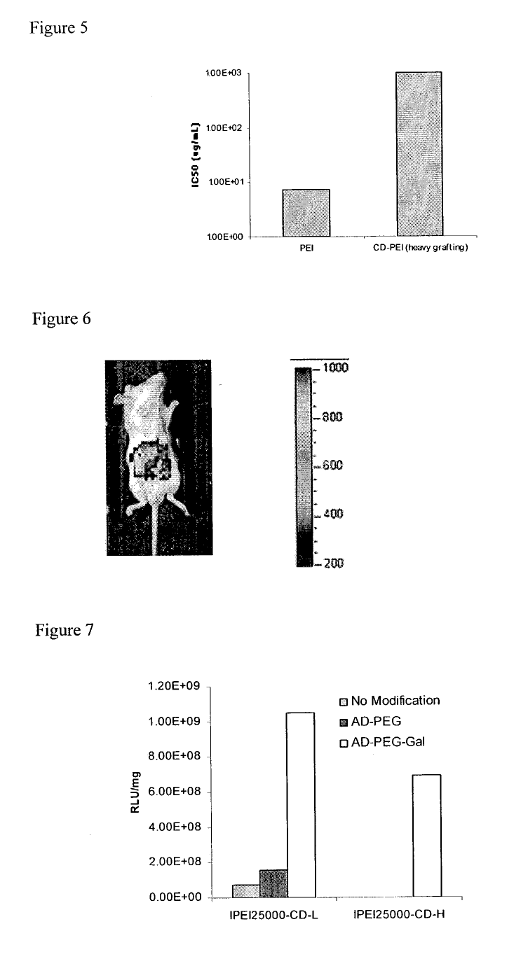

PC3 cells were plated at 60,000 cells/mL in 96 well plates (0.1 mL per well). After 24 hours, polymer solutions in media were added to the third column and diluted serially across the rows. The cells were incubated for 24 hours, after which they were washed with PBS and 50 μL of MTT (2 mg/mL in PBS) per well was added, followed by 150 μL of complete media per well. The wells were incubated for 4 hours. The solutions were then removed and 150 μL of DMSO was added. Adsorbance was then read at 540 nm. Results for branched CD-PEI are depicted in FIG. 5.

Toxicities of Various CD-PEI Polymers. Comparisons to Mannosylated-PEI (Man-JET-PEI)

The IC₅₀'s of cyclodextrin-grafted 1PEI and bPEI polymers in PC3 cells were determined by MTT assay. As a comparison, the IC₅₀ of mannosylated-PEI (man-JET-PEI) along with the parent PEI (JET-PEI), purchased from Polyplus Transfections (Illkirch, France), was determined for comparison. The IC₅₀ values were determined as follows:

PC3 cells were plated at 60,000 cells/mL in 96-well plates for 24 hours (0.1 mL per well). Polymers were added to the third column in complete and diluted serially across the rows. After 24 hours, the cells were washed with PBS and 50 μL of MTT (2 mg/mL in PBS) was added per well followed by 150 μL of complete media. The media was removed after 4 hour incubation and 150 μL of DMSO was added. Adsorbance was read at 540 nm.

The IC₅₀ values are shown in the chart below. Polymers are shown grouped in pairs (parent polymer and modified polymer) in the first column. The IC₅₀ value for each polymer is listed in the second column in μg/mL. The third column lists the decrease in toxicity by saccharide grafted, as calculated by the modified PEI IC₅₀ value divided by the parents PEI IC₅₀ value. The cyclodextrin-grafted PEIs have IC₅₀ values that are over forty times those of mannosylated PEI from Polyplus. In addition, modification with high grafting density results in a much higher increase in tolerability (90-fold vs. 20 fold) over parent polymers.

| Polymer | IC₅₀ (μg/mL) | Fold Increase |
|---|---|---|
| b-PEI25000 | 7.5 | |
| b-PEI25000-CD | 1000 | 133 |
| l-PEI25000 | 11 | |
| l-PEI25000-CD | 1000 | 90 |
| JET-PEI | 1.1 | |
| Man-JET-PEI | 23 | 20 |

### EXAMPLE 7

In vivo Delivery of DNA by Branched CD-PEI

Balb-C mice were injected with PEGylated CD-PEI polyplexes containing 200 μg of pGL3-CV (15:5:1 AD-PEG: CD-PEI: pGL3-CV by weight) by portal vein injection. Mice were anesthesized, injected with luciferin, and imaged using a Xenogen camera 4.5 hours after injection. Luciferase expression was observed in the liver, as indicated by light emission as shown in FIG. 6.

### EXAMPLE 8

Transfection of Galactosylated CD-PEI to Hepatoma Cells in vitro

CD-PEI based polyplexes (containing the α-luciferase plasmid) were modified by PEG-galactose and PEG by adding in AD-PEG₅₀₀₀-Galactose (adamantane-polyethylene glycol-galactose) or AD-PEG₅₀₀₀ during polyplex formulation (for more information on adamantane conjugates and inclusion complexes thereof, see PCT publication WO 02/49676). The adamantane from AD-PEG₅₀₀₀-Galactose or AD-PEG₅₀₀₀ forms inclusion complexes with the cyclodextrin and modifies the surface of the particles with PEG-galactose or PEG, respectively. These polyplexes were exposed to HepG2 cells, hepatoma cells expressing the asialoglycoprotein receptor. Polyplexes modified by galactose yielded a 10-fold increase in luciferase expression as shown in FIG. 7, indicating increased transfection by galactose-mediated uptake.

### EXAMPLE 9

Determination of Effect of CD-bPEI Cyclodextrin Loading on Transfection Efficiency

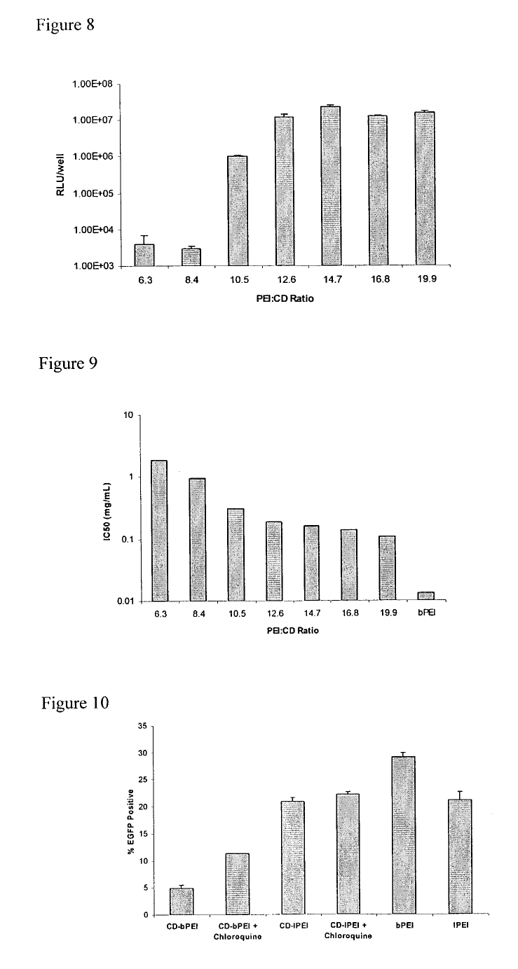

PC3 cells were plated at 50,000 cells/well in 24-well plates 24 hours before transfection. Immediately prior to transfection, cells in each well were rinsed once with PBS before the addition of 200 μL of Optimem (Invitrogen) containing polyplexes (1 μg of DNA complexed with polycation synthesized as described in Example 1 at 10 N/P). After 4 hours, transfection media was aspirated and replaced with 1 mL of complete media. After another 24 hours, cells were washed with PBS and lysed by the addition of 100 μL of Cell Culture Lysis Buffer (Promega, Madison, Wis.). Cell lysates were analyzed for luciferase activity with Promega's luciferase assay reagent. Light units were integrated over 10 s with a luminometer (Monolight 3010C, Becton Dickinson). High transfection was observed with PEI:CD ratios greater than 10 (see FIG. 8).

Determination of Effect of CD-bPEI Cyclodextrin Loading on Cell Toxicity

PC3 cells were plated in 96-well plates at 5,000 cells/well for 24 hours. Polymers were added to the third column and diluted serially across the rows. After another 24 hours, cells were washed with PBS and 50 μL of MTT (2 mg/mL in PBS) was added per well followed by 150 μL of complete media. Media was removed after 4 hours incubation at 37° C. and 150 μL of DMSO was added to dissolve the formazan crystals. Absorbance was read 540 nm to determine cell survival. All experiments were conducted in triplicate and averaged. Average absorbance was plotted versus polymer concentration and IC₅₀ values were determined by interpolation within the linear absorbance region. The tolerability of the polymers increases as more CD is grafted onto bPEI (see FIG. 9).

### EXAMPLE 10

Determination of Effect of CD-1PEI Cyclodextrin Loading on Cell Toxicity

The IC₅₀ of the CD-1PEI polymer to PC3 cells (with 8.4 PEI:CD, synthesis described in Example 3) was determined according to the procedure in Example 9 and compared with the IC₅₀ of the parent 1PEI polymer. The IC₅₀ of CD-1PEI (220 μg/mL) was 15 times greater than the IC₅₀ of 1PEI (15 μg/mL).

Determination of Effect of Chloroquine on Transfection Efficiency With CD-1PEI

PC3 cells were plated at 250,000 cells/well in 6-well plates. After 24 hours, the cells were transfected with 5 μg of pEGFP-luc plasmid assembled with polymer at N/P in 1 mL of Optimem (for some samples, Optimem containing 200 μM chloroquine was added). Four hours after transfection, media was removed and replaced with 5 mL of complete media. Cells were washed with PBS, trypsinized, and analyzed by flow cytometry for EGFP expression 48 hours after transfection. Grafting of cyclodextrin onto 1PEI at 8.4 PEI:CD does not affect transfection efficiency. Results are presented in FIG. 10.

### EXAMPLE 11

Formulation of CD-bPEI and CD-1PEI-based Particles

An equal volume of polycation (dissolved in water or D5W) is added to DNA (0.1 mg/mL in water). The polymer nitrogen to DNA phosphate ratio (N/P) is varied by changing the concentration of the polycation solution.

Electron Micrographs of CD-bPEI Particles

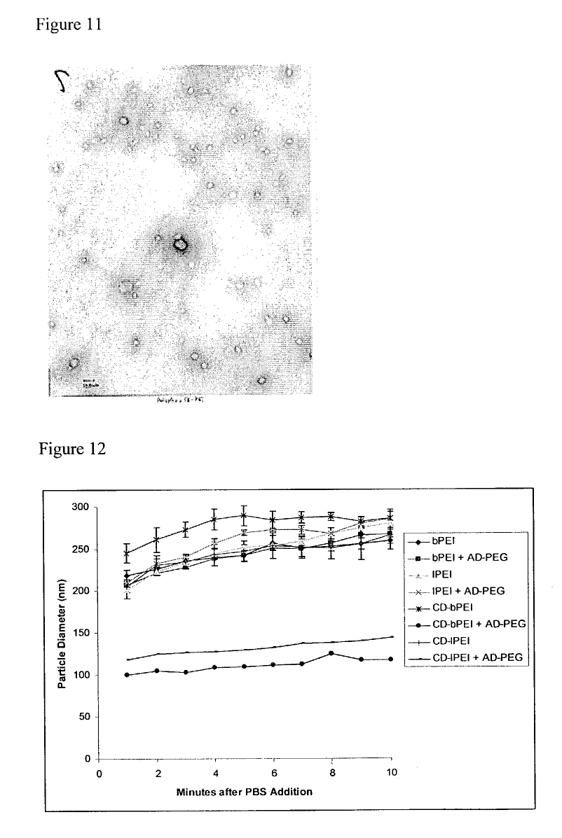

Polyplexes were formulated using CD-bPEI (12.6 PEI:CD ratio) at 10 N/P as described above. 5 μL of polyplexes were applied to 400-mesh carbon-coated copper grids for 45 seconds, after which excess liquid was removed by blotting with filter. Samples were negatively stained with 2% uranyl acetate for 45 seconds before blotting. The 400-mesh carbon-coated copper grids were glow-discharged immediately prior to sample loading. Images, as depicted in FIG. 11, were recorded using a Philips 201 electron microscope operated at 80 kV.

Particle Size and CD-bPEI and CD-1PEI Particles

Particles were formulated using CD-bPEI (12.6 PEI:CD ratio) at 10 N/P as described above and then diluted by the addition of 1.2 mL of water. Particle size was measured using a ZetaPals dynamic light scattering detector (Brookhaven Instrument Corporation). Three measurements were taken for each sample and data reported as average size.

| | Average Particle Diameter | Standard Deviation |
|---|---|---|
| Polymer | (nm) | (nm) |
| bPEI | 290 | 3 |
| lPEI | 115 | 2 |
| CD-bPEI | 96 | 1 |
| CD-lPEI | 93 | 1 |

Salt Stabilization of CD-bPEI and CD-1PEI Particles by the Addition of AD-PEG

Particles were formulated as described above and then diluted by the addition of 1.2 mL PBS. Particle size was monitored using a ZetaPals dynamic light scattering detector every minute for 10 minutes. Samples were run in triplicate and data reported as average size at each time point. The addition of AD-PEG helps to stabilization CD-bPEI and CD-1PEI particles against salt-induced aggregation. Addition of AD-PEG to bPEI and 1PEI particles has no affect on salt-induced aggregation. Results are presented in FIG. 12.

### EXAMPLE 12

Oligonucleotide Delivery With CD-bPEI and CD-1PEI Particles

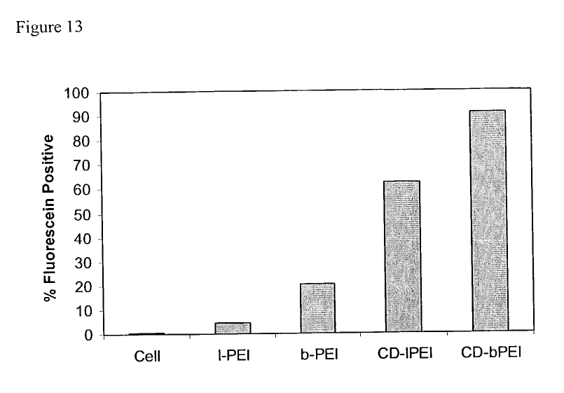

PC3 cells were plated at 2,000,000 cells/well in 6-well plates. After 24 hours, the cells were transfected with 5 μg of fluorescently-labeled oligonucleotide complexed with polycation at 10 N/P. After 15 minutes, cells were washed with PBS, cell scrub buffer, and trypsinized and analyzed by flow cytometry for uptake of the polyplexes. CD-bPEI (12.6 PEI:CD) and CD-1PEI (8.4 PEI:CD) are efficient at delivering oligos to cultured cells. Results are depicted in FIG. 13.

### EXAMPLE 13

In vivo Tolerability of CD-1PEI and CD-bPEI Polymers

Female, Balb/C mice were injected intravenously with CD-1PEI- and CD-bPEI-based polyplexes using a volume of 0.4 mL (D5W based solution) and injection speed of ˜0.2 ml/15 sec. Animals were sacrificed 24 hours after injection and blood collected for transaminase, creatinine, platelet and white blood cell analysis.

Groups:

| | | | |
|---|---|---|---|
| 1. | Control | | |
| 2. | CD-bPEI | 10 N/P | 0.1 mg DNA/mL |
| 3. | CD-bPEI | 10 N/P | 0.2 mg DNA/mL |
| 4. | CD-bPEI | 10 N/P | 0.3 mg DNA/mL |
| 5. | CD-lPEI | 10 N/P | 0.1 mg DNA/mL |
| 6. | CD-lPEI | 10 N/P | 0.2 mg DNA/mL |
| 7. | CD-lPEI | 10 N/P | 0.3 mg DNA/mL |

The maximum tolerable dose of CD-bPEI was determined to be 9 mg/kg (assuming 20 g mice, 0.1 mg DNA/mL dose). At the 0.2 mg DNA/mL dose, all animals survived but with depressed platelet counts.

The maximum tolerable dose of CD-1PEI was determined to be at least 36 mg/kg (assuming 20 g mice, 0.3 mg DNA/mL dose). No platelet depression or elevated liver enzyme levels was observed. In addition, all animals survived at the highest dose injected.

As a comparison, the LD₅₀ of 1PEI was determined to be ˜3-4 mg/kg (50% Balb/C mice died with an injection of 50 μg of DNA complexed with 1PEI at 10 N/P; Chollet et al. J Gene Medicine v4:84-91 (2002).

In vivo Expression With CD-1PEI Polyplexes Injected Into Xenograph Tumors

CD-1PEI particles were injected into tumors of Neuro2a tumor-bearing mice (120 μg DNA complexed with CD-1PEI at 10 N/P per mouse). After 48 hours, tumors were excised, homogenized and analyzed for luciferase expression. Average expression was determined to be: 2500 RLU/mg tissue.

### EXAMPLE 14

Synthesis of Galactose-bPEI

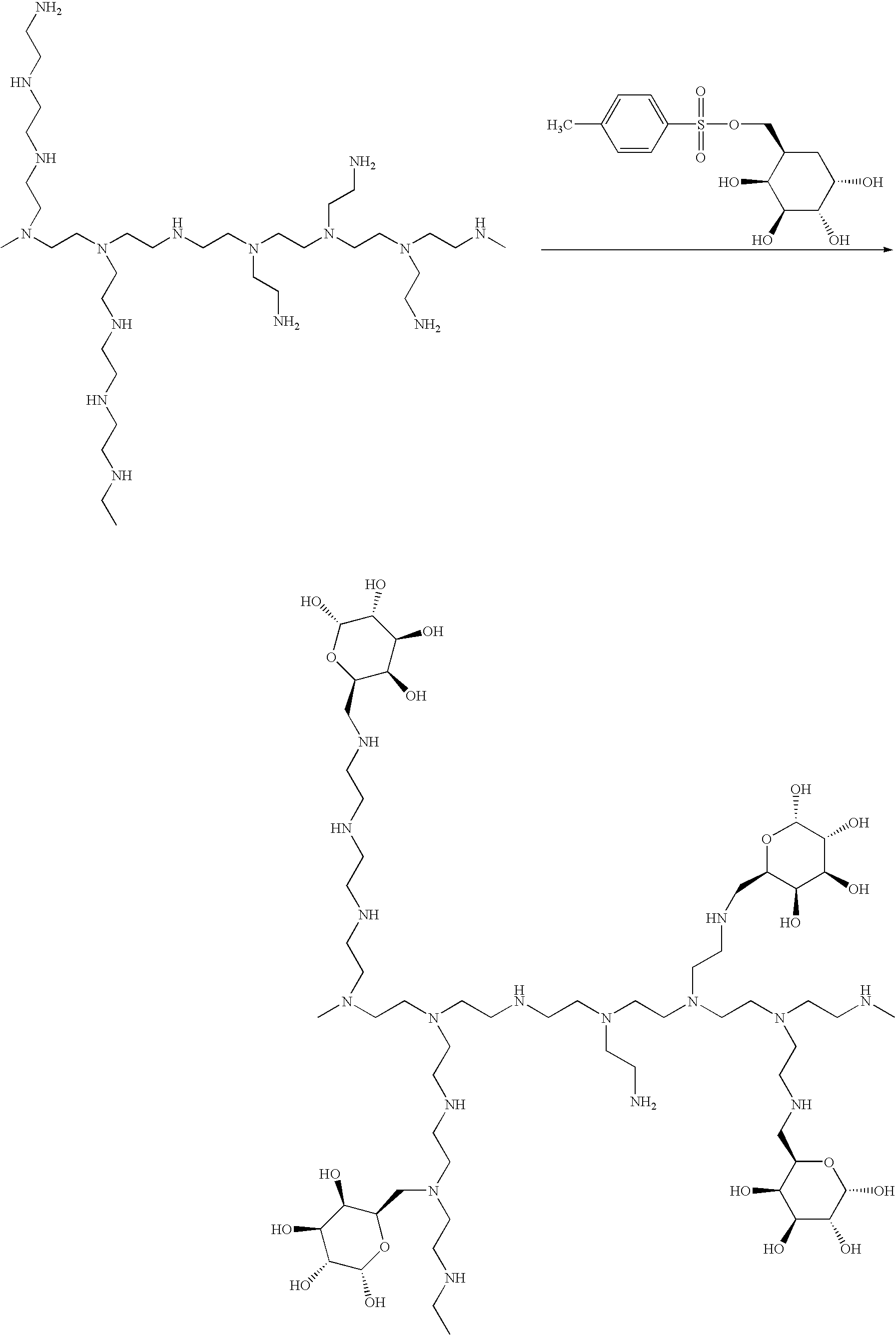

Protocol:

a. Synthesis of Tosyl-Galactose:

p-Toluenesulfonylchloride (5.8 g, 30.5 mmol, Acros) in anhydrous pyridine (10 mL) was added dropwise to a solution of D-galactose (5 g, 27.8 mmol, Aldrich) in anhydrous pyridine (50 mL) at 0° C. The solution was stirred for 4 h at room temperature. The reaction mixture was then quenched with MeOH (2 mL), diluted with 75 mL of CHCl₃, and washed twice with ice-cold water (50 mL). The organic phase was dried under reduced pressure. The residue was subjected to C8 reversed-phase column chromatography using a gradient elution of 0-50% acetonitrile in water. Fractions were analyzed on a Beckman Coulter System Gold HPLC system equipped with a UV 168 Detector, an Evaporative Light Scattering (ELS) Detector and a C18 reversed-phase column (Alltech) using an acetonitrile/H₂O gradient as eluant at 0.7 mL/min flow rate. The appropriate fractions were combined and evaporated to dryness. This procedure gave the tosyl-galactose as confirmed by mass spectroscopy: Electrospray Ionization: 357.1 [M+Na]⁺, 690.7 [2M+Na]⁺.

b. Synthesis of Galactose-bPEI with different galactose loading

Low loading: Branched PEI_{25,000} (64.9 mg, 0.0026 mmol, Aldrich, MW 25,000) and tosyl-galactose (13 mg, 0.039 mmol) was dissolved in 22 mL of H₂O/DMSO (5/95). The solution was stirred at 70° C. for 3 days. The solution was then transferred to a Spectra/Por MWCO 10,000 membrane and dialyzed against water for 6 days. Water was then removed by lyophilization to afford a slightly colored solid. Galactose/PEI ratio was calculated based on the proton integration of ¹H-NMR (Varian 300 MHz, D₂O).

High loading: Branched PEI_{25,000} (64.9 mg, 0.0026 mmol, Aldrich, MW 25,000) and tosyl-galactose (130 mg, 0.39 mmol) was dissolved in 22 mL of H₂O/DMSO (5/95). The solution was stirred at 70° C. for 3 days. The solution was then transferred to a Spectra/Por MWCO 10,000 membrane and dialyzed against water for 6 days. Water was then removed by lyophilization to afford a slightly colored solid. Galactose/PEI ratio was calculated based on the proton integration of ¹H NMR (Varian 300 MHz, D₂O).

### EXAMPLE 15

Synthesis of Galactose-1PEI

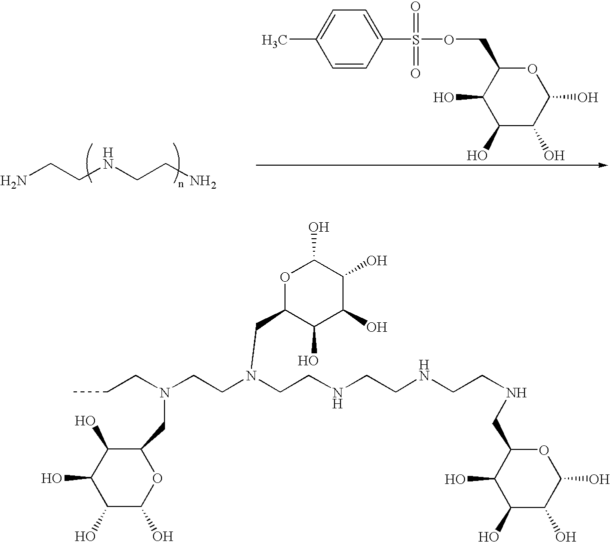

Protocol:

Low loading: Linear PEI_{25,000} (100 mg, 0.004 mmol, Polyscience, MW 25,000) and tosyl-galactose (20 mg, 0.06 mmol) were dissolved in 7.2 mL of DMSO. The solution was stirred at 70° C. for 6 days. The solution was then transferred to a Spectra/Por MWCO 10,000 membrane and dialyzed against water for 6 days. Water was then removed by lyophilization to afford a slightly colored solid. Galactose/PEI ratio was calculated based on the proton integration of ¹H NMR (Varian 300 MHz, D₂O).

High loading: Linear PEI_{25,000} (100 mg, 0.004 rnmol, Polyscience, MW 25,000) and tosyl-galactose (200 mg, 0.6 mmol) was dissolved in 7.2 mL of DMSO. The solution was stirred at 70° C. for 6 days. The solution was then transferred to a Spectra/Por MWCO 10,000 membrane and dialyzed against water for 6 days. Water was then removed by lyophilization to afford a slightly colored solid. Galactose/PEI ratio was calculated based on the proton integration of ¹H NMR (Varian 300 MHz, D₂O).

All of the above-cited references and publications are hereby incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A polymer comprising poly(ethylenimine) coupled to cyclodextrin moieties.

2. The polymer of claim 1, wherein the poly(ethylenimine) is a branched polymer.

3. The polymer of claim 1, wherein the poly(ethylenimine) is a linear polymer.

4. The polymer of claim 1, wherein the cyclodextrin moieties are covalently coupled to the poly(ethylenimine).

5. The polymer of claim 1, wherein the poly(ethylenimine) is covalently coupled to guest moieties that form inclusion complexes with cyclodextrin, and the carbohydrate moieties are coupled to the poly(ethylenimine) through inclusion complexes of cyclodextrins with the guest moieties.

6. The polymer of claim 1, wherein the polymer has a structure of the formula: 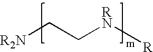
wherein R represents, independently for each occurrence, H, lower alkyl, a moiety including a cyclodextrin moiety, or 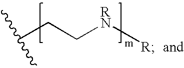
m, independently for each occurrence, represents an integer greater than 10.

7. The polymer of claim 1, wherein the ratio of ethylenimine units to cyclodextrin moieties in the polymer is between about 4:1 and 20:1.

8. The polymer of claim 1, wherein the ratio of ethylenimine units to cyclodextrin moieties in the polymer is between about 9:1 and 20:1.

9. A polymer comprising a structure of the formula: 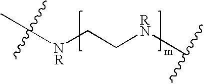
wherein R represents, independently for each occurrence, H, lower alkyl, a moiety including a carbohydrate moiety, or 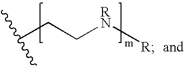
m, independently for each occurrence, represents an integer greater than 10,
wherein about 3-15% of the occurrences of R represent a moiety including a carbohydrate moiety other than a galactose or mannose moiety.

10. A polymer of claim 9, wherein the carbohydrate moieties include cyclodextrin moieties.

11. A polymer of claim 9, wherein the carbohydrate moieties consist essentially of cyclodextrin moieties.

12. A polymer of claim 9, wherein about 3-25% of the occurrences of R represent a moiety including a cyclodextrin moiety.

13. A composition comprising a polymer of claim 1 and a nucleic acid.

14. A method for transfecting a cell with a nucleic acid, comprising contacting the cell with a composition of claim 13.

15. A kit comprising a polymer of claim 1 and instructions for combining the polymer with a nucleic acid for transfecting cells with the nucleic acid.

16. A method of conducting a pharmaceutical business, comprising providing a distribution network for selling a polymer of claim 1, and providing instruction material to patients or physicians for using the polymer to treat a medical condition.

17. A method of conducting a pharmaceutical business, comprising providing a distribution network for selling a kit of claim 15, and providing instruction material to patients or physicians for using the kit to treat a medical condition.

18. A composition comprising a polymer of claim 9 and a nucleic acid.

19. A method for transfecting a cell with a nucleic acid, comprising contacting the cell with a composition of claim 18.

20. A kit comprising a polymer of claim 9 and instructions for combining the polymer with a nucleic acid for transfecting cells with the nucleic acid.

21. A method of conducting a pharmaceutical business, comprising providing a distribution network for selling a polymer of claim 9, and providing instruction material to patients or physicians for using the polymer to treat a medical condition.

22. A method of conducting a pharmaceutical business, comprising providing a distribution network for selling a kit of claim 20, and providing instruction material to patients or physicians for using the kit to treat a medical condition.

23. Particles comprising a polymer of claim 1 and having a diameter between 50 and 1000 nm.

24. Particles of claim 23, further comprising a nucleic acid.

25. Particles of claim 23, further comprising polyethylene glycol chains coupled to the polymer through inclusion complexes with the cyclodextrin moieties.

26. Particles comprising a polymer of claim 10 and having a diameter between 50 and 1000 nm.

27. Particles of claim 26, further comprising a nucleic acid.

28. Particles of claim 26, further comprising polyethylene glycol chains coupled to the polymer through inclusion complexes with the cyclodextrin moieties.

29. A polymer comprising linear poly(ethylenimine) coupled to carbohydrate moieties.
